(19) United States
(12) Patent Application Publication
(10) Pub. No.: US 2003/0152630 A1
(43) Pub. Date: Aug. 14, 2003

(54) PEG-POE, PEG-POE-PEG, and POE-PEG-POE block copolymers

(76) Inventors: Steven Y. Ng, San Francisco, CA (US); Jorge Heller, Woodside, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/139,114

(22) Filed: May 2, 2002

Related U.S. Application Data
(60) Provisional application No. 60/386,201, filed on May 11, 2001.

(51) Int. Cl. A61K 9/14; C08L 33/04

(52) U.S. Cl. 424/486; 525/94

(57) ABSTRACT

PEG-POE, PEG-POE-PEG, and POE-PEG-POE block copolymers have both hydrophilic and hydrophobic blocks. They form micelles in aqueous solution, making them suitable for encapsulation or solubilization of hydrophobic or water-insoluble materials; and they also form bioerodible matrices for the sustained release of active agents, especially when the POE block(s) contain at least one unit containing an α-hydroxy acid.

### CROSS-REFERENCE TO RELATED APPLICATION

This application claims the priority under 35 USC 119(e) of Provisional Application No. 60/ ______ filed May 11, 2001 (application Ser. No. 09/854,150, filed May 11, 2001, for which a petition to convert to a provisional application was filed on Mar. 29, 2002).

### BACKGROUND OF THE INVENTION

1. Field of the Invention

This invention relates to poly(ethylene glycol)-poly(ortho ester), poly(ethylene glycol)-poly(ortho ester)-poly(ethylene glycol), and poly(ortho ester)-poly(ethylene glycol)-poly(ortho ester) block copolymers.

2. Discussion of the Related Art

Micellar System for Tumor Targeting

One of the major problems in treating cancer is the difficulty of achieving a sufficient concentration of an anticancer agent in the tumor. This is due to the toxicity, sometimes extreme, of such agents which severely limits the amounts that can be used. However, a major discovery in cancer chemotherapy has been the so-called EPR (enhanced permeation and retention) effect. The EPR effect is based on the observation that tumor vasculature, being newly formed vasculature, has an incompletely formed epithelium and is much more permeable than established older vasculature which is essentially impermeable to large molecules. Further, lymphatic drainage in tumors is very poor thus facilitating retention of anticancer agents delivered to the tumor.

The EPR effect can be used in cancer targeting by using delivery systems containing anticancer drugs that are too large to permeate normal vasculature, but which are small enough to permeate tumor vasculature, and two approaches have been developed. In one approach, a water-soluble polymer is used that contains an anticancer drug chemically bound to the polymer via a hydrolytically labile linkage. Such drug-polymer constructs are injected intravenously and accumulate in the tumors, where they are internalized by the cells via endocytosis and released in the lysosomal compartment of the cell via enzymatic cleavage of the labile bond attaching the drug to the polymer. Two disadvantages of this approach are that, first, nondegradable, water-soluble polymers have been used, and this requires a tedious fractionation of the polymer to assure that the molecular weight of the polymer is below the renal excretion threshold, and, second, the drug must be chemically attached to the polymer, which in effect creates a new drug entity with consequent regulatory hurdles that must be overcome. The use of polymer conjugates in cancer diagnosis and treatment is discussed in Duncan et al., “The role of polymer conjugates in the diagnosis and treatment of cancer”, S. T. P. Pharma Sciences, 6(4), 237-263 (1996), and an example of an alginate-bioactive agent conjugate is given in U.S. Pat. No. 5,622,718.

An alternate approach has been described. In this approach, an AB or ABA block copolymer is prepared where the B-block is hydrophobic and the A-block is hydrophilic. When such a material is placed in water, it will self-assemble into micelles with a hydrophobic core and a hydrophilic shell surrounding the core. Such micelles have a diameter of about 100 nm, which is large enough that when they are injected intravenously, the micelles can not leave the normal vasculature, but they are small enough to leave the vasculature within tumors. Further, a 100 nm diameter is too small to be recognized by the reticuloendothelial system, thus enhancing micelle lifetime within the blood stream. Additionally, when the hydrophilic block is poly(ethylene glycol), further enhancement of circulation time is noted, as has been observed with “stealth” liposomes. The use of block copolymer micelles is reviewed in Kwon et al., “Block copolymer micelles as long-circulating drug delivery vehicles”, Adv. Drug Delivery Rev., 16, 295-309 (1995).

U.S. Pat. Nos. 5,412,072; 5,449,513; 5,510,103; and 5,693,751 describe block copolymers useful as micellar delivery systems where the hydrophilic block is polyethylene glycol and the hydrophobic blocks are various derivatives of poly(aspartic acid), poly(glutamic acid) and polylysine. U.S. Pat. Nos. 5,412,072 and 5,693,751 describe an approach where drugs have been chemically attached to the hydrophobic segment; while U.S. Pat. Nos. 5,449,513 and 5,510,103 describe an approach where hydrophobic drugs have been physically entrapped within the hydrophobic portion of the micelle. This latter approach is clearly preferable because no chemical modification of the drug is necessary.

Bioerodible Block Copolymer Matrix for Controlled Drug Delivery

In AB, ABA, or BAB block copolymers comprising a hydrophilic A block and a hydrophobic B block, the A and B blocks are incompatible and on a microscopic scale will phase-separate. This phase separation imparts unique and useful thermal properties to the material.

There is considerable prior art in the development of block copolymers comprised of poly(ethylene glycol) and bioerodible hydrophobic segments such as poly(L-lactic acid), poly(L-lactic-co-glycolic acid) copolymers and poly(ε-caprolactone), and discussion of the use as drug delivery agents. For example, see Wolthuis et al., “Synthesis and characterization of poly(ethylene glycol) poly-L-lactide block copolymers”, Third Eur. Symp. Controlled Drug Delivery, 271-276 (1994), Youxin et al., “Synthesis and properties of biodegradable ABA triblock copolymers . . . ”, J. Controlled Release, 27, 247-257 (1993), and U.S. Pat. No. 5,133,739.

Poly(ortho esters) are known as potential vehicles for sustained release drug delivery. See, for example, Heller, “Poly (Ortho Esters)”, Adv. Polymer Sci., 107, 41-92 (1993), and references cited therein, and U.S. Pat. Nos. 4,304,767, 4,946,931, 4,957,998, and 5,968,543.

U.S. Pat. No. 5,939,453 describes block copolymers prepared from polyethylene glycols and certain poly(ortho esters).

These and other documents referred to in this application are incorporated into this application by reference.

### SUMMARY OF THE INVENTION

In a first aspect, this invention is block copolymers of formula X, formula Y, and formula Z:

R¹—[OCH₂CH₂]_{f}—[POE]_{g}—H (X),

R¹—[OCH₂CH₂]_{f}—[POE]_{g}—[OCH₂CH₂]ₕ—OR² (Y),

H—A—[POE]_{g}—[OCH₂CH₂]ₕ—[POE]ⱼ—H (Z),

where:

R¹ is C₁₋₄ alkyl;

R² is C₁₋₄ alkyl;

f and h are independently an integer from 2 to 1000;

g and j are independently an integer from 2 to 200;

POE is a poly(ortho ester) of formula I or formula II:

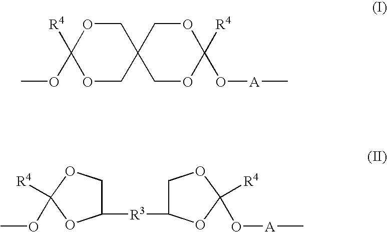

where:

R³ is a bond, —(CH₂)ₐ—, or —(CH₂)_{b}—O—(CH₂)_{c}—; where a is an integer of 1 to 10, and b and c are independently integers of 1 to 5;

R⁴ is a C₁₋₄ alkyl; and

each A is R⁵, R⁶, or R⁷, where

R⁵ is:

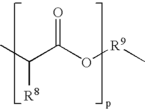

where:

p is an integer of 1 to 20;

R⁸ is hydrogen or C₁₋₄ alkyl; and

R⁹ is:

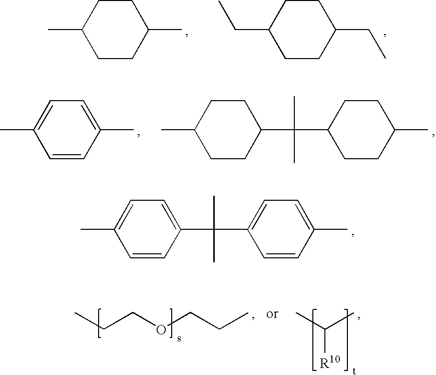

where:

s is an integer of 0 to 30;

t is an integer of 2 to 20; and

R¹⁰ is hydrogen or C₁₋₄ alkyl;

R⁶ is:

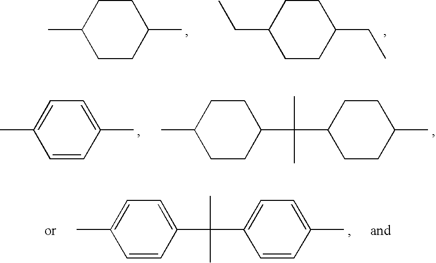

R⁷ is a diol containing at least one functional group independently selected from amide, imide, urea, and urethane groups;

where A is R⁷ in at least 0.1 mol % of the POE units when the POE is of formula I.

In a second aspect, this invention is a micellar pharmaceutical composition for the delivery of a hydrophobic or water-insoluble active agent, comprising the active agent physically entrapped within but not covalently bonded to a drug carrier comprising a block copolymer of formula X, formula Y, or formula Z, or a mixture thereof.

In a third aspect, this invention is a composition for the sustained release of an active agent, comprising the active agent dispersed in a matrix comprising a block copolymer of formula X, formula Y, or formula Z, or a mixture thereof.

In a fourth aspect, this invention is a process for the preparation of a block copolymer of formula X, formula Y, or formula Z, as described in the “Detailed Description of the Invention”.

### DETAILED DESCRIPTION OF THE INVENTION

Definitions

Unless defined otherwise in this specification, all technical and scientific terms are used herein according to their conventional definitions as they are commonly used and understood by those of ordinary skill in the art of synthetic and pharmaceutical chemistry.

“Active agent” includes any compound or mixture of compounds which produces a beneficial or useful result. Active agents are distinguishable from such components as vehicles, carriers, diluents, lubricants, binders and other formulating aids, and encapsulating or otherwise protective components. Examples of active agents are pharmaceutical, agricultural or cosmetic agents. Suitable pharmaceutical agents include locally or systemically acting pharmaceutically active agents which may be administered to a subject by topical or intralesional application (including, for example, applying to abraded skin, lacerations, puncture wounds, etc., as well as into surgical incisions) or by injection, such as subcutaneous, intradermal, intramuscular, intraocular, or intra-articular injection. Examples of these agents include, but not limited to, anti-infectives (including antibiotics, antivirals, fungicides, scabicides or pediculicides), antiseptics (e.g., benzalkonium chloride, benzethonium chloride, chlorhexidine gluconate, mafenide acetate, methylbenzethonium chloride, nitrofurazone, nitromersol and the like), steroids (e.g., estrogens, progestins, androgens, adrenocorticoids, glucocorticoids, and the like), therapeutic polypeptides (e.g., insulin, erythropoietin, morphogenic proteins such as bone morphogenic protein, and the like), analgesics and anti-inflammatory agents (e.g., aspirin, ibuprofen, naproxen, ketorolac, COX-1 inhibitors, COX-2 inhibitors, and the like; and the anti-inflammatory steroids), cancer chemotherapeutic agents (e.g., mechlorethamine, cyclophosphamide, fluorouracil, thioguanine, carmustine, lomustine, melphalan, chlorambucil, streptozocin, methotrexate, vincristine, bleomycin, vinblastine, vindesine, dactinomycin, daunorubicin, doxorubicin, tamoxifen, and the like), narcotics (e.g., morphine, meperidine, codeine, and the like), local anesthetics (e.g., the amide- or anilide-type local anesthetics such as bupivacaine, dibucaine, mepivacaine, procaine, lidocaine, tetracaine, and the like), antiangiogenic agents (e.g., combrestatin, contortrostatin, anti-VEGF agents, and the like), polysaccharides, vaccines, antigens, DNA and other polynucleotides, antisense oligonucleotides, and the like. The present invention may also be applied to other locally acting active agents, such as astringents, antiperspirants, irritants, rubefacients, vesicants, sclerosing agents, caustics, escharotics, keratolytic agents, sunscreens and a variety of dermatologics including hypopigmenting and antipruritic agents. The term “active agents” further includes biocides such as fungicides, pesticides, and herbicides, plant growth promoters or inhibitors, preservatives, disinfectants, air purifiers and nutrients.

“Alkyl” denotes a linear saturated hydrocarbyl having from one to the number of carbon atoms designated, or a branched or cyclic saturated hydrocarbyl having from three to the number of carbon atoms designated (e.g., C₁-C₄ alkyl). Examples of alkyl include methyl, ethyl n-propyl, isopropyl, cyclopropyl, n-butyl, t-butyl, cyclopropylmethyl, and the like.

“Bioerodible”, “biodegradable”, and the like terms refer to the degradation, disassembly or digestion of the polymer by action of a biological environment, including the action of living organisms, and most notably at physiological pH and temperature. A principal mechanism for bioerosion of the copolymers of the present invention is hydrolysis of linkages between and within the poly(ortho ester) blocks of the copolymer.

“Comprising” is an inclusive term interpreted to mean containing, embracing, covering or including the elements listed following the term, but not excluding other unrecited elements.

“Controlled release”, “sustained release”, and similar terms mean a mode of active agent delivery that occurs when the active agent is released from the vehicle or carrier at an ascertainable and controllable rate over a period of time, rather than dispersed immediately upon ingestion or application. Controlled or sustained release may extend for hours, days or months, and may vary as a function of numerous factors. In the present invention, an important determinant of the rate of delivery is the rate of hydrolysis of the linkages between and within the copolymer. The rate of hydrolysis in turn may be controlled by the composition of the copolymer and the number of hydrolysable bonds in the copolymer. Other factors include particle size, particle composition, particle hydration, acidity of the medium (either internal or external to the matrix), solubility of the active agent in the matrix and molecular weight and charge density of the active agent.

“Delivery vehicle” denotes a composition which has the functions including transporting an active agent to a site of interest, controlling the rate of access to, or release of, the active agent by sequestration or other means, and facilitating the application of the agent to the region where its activity is needed.

“Matrix” means the physical structure of the copolymer. Solid matrices essentially retain the active agent in a manner preventing release of the agent until the copolymer erodes or decomposes.

“PEG” means polyethylene glycol, H—[OCH₂CH₂]_{f}—OH, with a numerical suffix indicating the nominial number average molecular weight, Mₙ. Unless the context requires otherwise, “PEG” also includes polyethylene glycol mono(C₁-C₄ alkyl) ethers, R—[OCH₂CH₂]_{f}—OH, where R is C₁-C₄ alkyl, sometimes referred to as “RPEG”.

“POE” means a poly(ortho ester).

“Sequestration” means the confinement or retention of an active agent within the internal spaces of a copolymer matrix. Sequestration of an active agent within the matrix may limit the toxic effect of the agent, prolong the time of action of the agent in a controlled manner, permit the release of the agent in a precisely defined location in an organism, or protect an unstable agent against the action of the environment.

A “therapeutically effective amount” means the amount that, when administered to an animal for treating a disease, is sufficient to effect treatment for that disease.

“Treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). For the purposes of this invention, a “disease” includes pain and/or inflammation.

A “unit” denotes an individual segment of a poly(ortho ester) chain, which consists of the residue of a diketene acetal molecule and the residue of a polyol.

An “α-hydroxy acid containing” unit denotes a unit where A is R⁵, i.e. in which the polyol is prepared from an α-hydroxy acid or cyclic diester thereof and a diol of the formula HO—R⁵—OH. The fraction of the poly(ortho ester) that is α-hydroxy acid containing units affects the rate of hydrolysis (or bioerodibility) of the poly(ortho ester), and in turn, the release rate of the active agent.

“Vehicle” and “carrier” mean an ingredient that is included in a composition such as a pharmaceutical or cosmetic preparation for reasons other than a therapeutic or other biological effect. Functions served by vehicles and carriers include transporting an active agent to a site of interest, controlling the rate of access to, or release of, the active agent by sequestration or other means, and facilitating the application of the agent to the region where its activity is needed. The copolymers of this invention may serve as vehicles for the sustained release of active agents.

Ranges given, such as temperatures, times, sizes, and the like, should be considered approximate, unless specifically stated.

Ingredient names are taken from the International Cosmetic Ingredient Handbook, 3rd edition, 1995.

The Block Copolymers of this Invention

In a first aspect, this invention provides block copolymers of formula X, formula Y, and formula Z:

R¹—[OCH₂CH₂]_{f}—[POE]_{g}—H (X),

R¹—[OCH₂CH₂]_{f}—[POE]_{g}—[OCH₂CH₂]ₕ—OR² (Y),

H—A—[POE]_{g}—[OCH₂CH₂]ₕ—[POE]ⱼ—H (Z),

where:

R¹ is C₁₋₄ alkyl;

R² is C₁₋₄ alkyl;

f and h are independently an integer from 2 to 1000;

g and j are independently an integer from 2 to 200;

POE is a poly(ortho ester) of formula I or formula II:

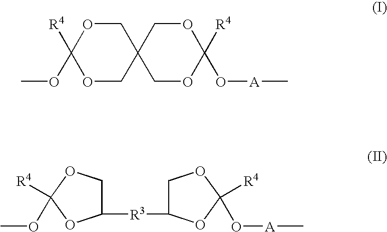

where:

R³ is a bond, —(CH₂)ₐ—, or —(CH₂)_{b}—O—(CH₂)_{c}—; where a is an integer of 1 to 10, and b and c are independently integers of 1 to 5;

R⁴ is a C₁₋₄ alkyl; and

A is R⁵, R⁶, or R⁷, where

R⁵ is:

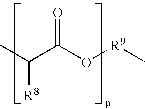

where:

p is an integer of 1 to 20;

R⁸ is hydrogen or C₁₋₄ alkyl; and

R⁹ is:

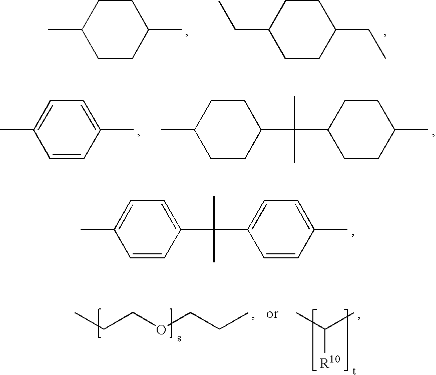

where:

s is an integer of 0 to 30;

t is an integer of 2 to 20; and

R¹⁰ is hydrogen or C₁₋₄ alkyl;

R⁶ is:

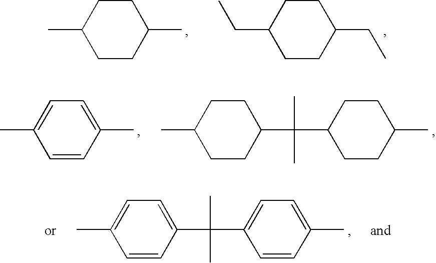

R⁷ is a diol containing at least one functional group independently selected from amide, imide, urea, and urethane groups;

where A is R⁷ in at least 0.1 mol % of the POE units when the poly(ortho ester) is of formula I.

The copolymers are AB (formula X), ABA (formula Y), and BAB (formula Z) block copolymers in which the A blocks are hydrophilic poly(ethylene glycol) and the B blocks are hydrophobic poly(ortho ester). Within these, the poly(ortho ester) blocks are composed of alternating residues of a diketene acetal and a diol.

The properties of the copolymers, including both the mechanophysical properties and the bioerodibility, are determined by the type of the copolymer, whether AB diblock, ABA triblock, or BAB triblock, the length of the PEG. and POE blocks, and the diol(s) used in the POE blocks (in particular, the proportion of diol of the general formula HO—R⁵—OH used in the POE blocks).

Preferred polymers are those in which one or more of the following are true:

(1) f and h are independently an integer from 10 to 500, especially from 50 to 250, for example 100, for micellar delivery; and f and h are independently an integer from 50 to 1000, especially from 100 to 1000, for example from 250 to 1000, for bioerodible matrices; and f and h are preferably the same if both are present;

(2) g and j are independently an integer from 5 to 100, especially 10 to 50, for example 15, for micellar delivery; and g and j are independently an integer from 10 to 200, especially from 20 to 200, for example from 50 to 200, for bioerodible matrices; and g and j are preferably the same if both are present;

(3) R⁴ is ethyl;

(4) R¹ and R² are methyl;

(5) R⁶ is 1,4-cyclohexanedimethanol;

(6) the proportion of POE units where A is R⁵ is from 0 to 10%

(7) in each R⁵ group p is 1 or 2 and R⁸ is hydrogen or methyl; and

(8) at least 0.1%, preferably at least 10%, more preferably at least 50%, especially at least 90%, and more especially 100% of the POE units are of formula II.

While a block copolymer having any one of these preferences listed above is preferred over a block copolymer not having that preference, the block copolymers will be more preferred the greater the number of preferences met.

Because of the polymeric character of these molecules, the number of repeating units within the blocks, f, g, h, and j necessarily represent averages of distributions rather than exact numbers; and in particular, when f and h or g and j are described as being the same, this indicates that the average values of f and h, or of g and j, should be approximately the same. Similarly, the lengths of other polymeric chains, such as the poly(ethylene glycol) of R⁹; of the long chain diol of R⁹; and of the poly(α-hydroxy acid) group within R⁵ necessarily represent averages of distributions rather than exact numbers.

The Starting Materials

Polyethylene glycols, and polyethylene glycol lower alkyl ethers of various chain lengths (molecular weights) are available from a number of sources, including Aldrich Chemical Company, Inc., Milwaukee, Wis., and Shearwater Polymers, Huntsville, Ala.

The preparation of the diketene acetals of the types of formula III and formula IV

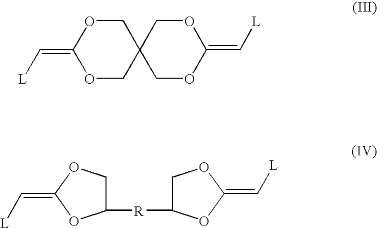

where L is hydrogen or a C₁₋₃ alkyl, is disclosed in U.S. Pat. Nos. 4,304,767, 4,532,335, and 5,968,543; and Crivello et al., J. Polymer Sci., Part A—Polymer Chemistry, 34, 3091-3102 (1996), and will be known to a person of ordinary skill in the art. A typical method is the condensation of a bis(diol) of formula V (i.e. pentaerythritol) or formula VI:

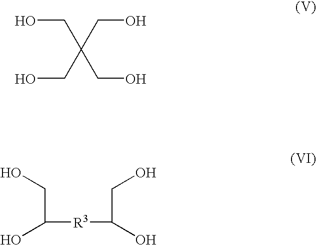

with two equivalents of a 2-halocarboxaldehyde dialkyl acetal, such as 2-bromoacetaldehyde diethyl acetal, followed by dehydrohalogenation to give the diketene acetal. The condensation of a glycol with diethylbromoacetals is described in Roberts et al., J. Am. Chem. Soc., 80, 1247-1254 (1958), and dehydrohalogenation is described in Beyerstedt et al., J. Am. Chem. Soc., 58, 529-553 (1936).

The diketene acetals may also be prepared by the isomerization of divinyl acetals. Thus, for example, 3,9-di(ethylidene)-2,4,8,10-tetraoxaspiro[5.5]undecane may be prepared by the isomerization of 3,9-divinyl-2,4,8,10-tetraoxaspiro[5.5]undecane, using n-butyllithium in ethylenediamine. The isomerization of the double bond is described in Corey et al., J. Org. Chem., 38, 3224 (1973). The divinyl acetals may be prepared by the condensation of the bis(diol) of formula V or formula VI with two equivalents of a vinylic aldehyde, such as acrolein or crotonaldehyde, or their dialkyl acetals, such as acrolein dimethyl acetal, and such condensation reactions are well known. Thus, for example, 2,2′-divinyl-4,4′-bi(1,3-dioxolanyl) is prepared from the reaction of erythritol with acrolein in benzene/p-toluenesulfonic acid, and is subsequently isomerized to 2,2′-diethylidene-4,4′-bi(1,3-dioxolanyl) with tris(triphenylphosphine)ruthenium (II) chloride.

The bis(diol) of formula VI where R³ is a bond is erythritol. The bis(diol) of formula VI where R³ is —(CH₂)ₐ— may be prepared by the oxidation of an α,ω-diene, such as 1,3-butadiene, 1,4-pentadiene, or 1,5-hexadiene, with an oxidizing reagent such as osmium tetroxide/hydrogen peroxide, or by other methods known in the art, to give the bis(diol). The bis(diol) of formula VI where R³ is —(CH₂)_{b}—O—(CH₂)_{c}— may be prepared by the reaction of an ω-hydroxy-α-olefin, such as allyl alcohol, with an ω-haloalkyloxirane, such as epichlorohydrin, to form an ω-epoxy-α-olefin with the backbone interrupted by an oxygen atom, such as 2-allyloxymethyloxirane, which is then oxidized with an oxidizing reagent such as osmium tetroxide/hydrogen peroxide, or by other methods known in the art, to give the bis(diol).

The diols of the formulae HO—R⁵—OH, HO—R⁶—OH, and HO—R⁷—OH are prepared according to methods known in the art, and as described, for example, in U.S. Pat. Nos. 4,549,010 and 5,968,543. Some of the diols are commercially available. The diol of the formula HO—R⁵—OH that comprises a polyester moiety may be prepared by reacting a diol of the formula HO—R⁹—OH with between 0.5 and 10 molar equivalents of a cyclic diester of an α-hydroxy acid, such as lactide or glycolide, and allowing the reaction to proceed at 100-200° C. for about 12 hours to about 48 hours. Although particular solvents are not required for this reaction, organic solvents such as dimethylacetamide, dimethylsulfoxide, dimethylformamide, acetonitrile, pyrrolidone, tetrahydrofuran, and methylbutyl ether may be used.

Diols of the formula HO—R⁷—OH include diols where R⁷ is of the form R′CONR″R′ (amide), R′CONR″COR′ (imide), R′NR″CONR″R′ (urea), and R′OCONR″R′ (urethane), where each R′ is independently an aliphatic, aromatic, or aromatic/aliphatic straight or branched chain hydrocarbyl, especially a straight or branched chain alkyl of 2 to 22 carbon atoms, especially 2 to 10 carbon atoms, and more especially 2 to 5 carbon atoms, and each R″ is hydrogen or C₁₋₆ alkyl, especially hydrogen or methyl, more especially hydrogen. Some representative diols of the formula HO—R⁷—OH include N,N′-bis-(2-hydroxyethyl)terephthalamide, N,N′-bis-(2-hydroxyethyl)pyromellitic diimide, 1,1′-methylene-di-(p-phenylene)-bis-[3-(2-hydroxyethyl)urea], N,N′-bis-(2-hydroxyethyl)oxamide, 1,3-bis(2-hydroxyethyl)urea, 3-hydroxy-N-(2-hydroxyethyl)propionamide, 4-hydroxy-N-(3-hydroxypropyl)butyramide, and bis(2-hydroxyethyl)ethylenedicarbamate. These diols are known to the art in reported syntheses and may are commercially available. Representative diols of the formula HO—(CH₂)ₙ—NHCO—(CH₂)ₘ—OH where n is an integer of 2 to 6 and m is an integer of 2 to 5 are made by the reaction of 2-aminoethanol, 3-aminopropanol, 4-aminobutanol, 5-aminopentanol, or 6-aminohexanol with β-propiolactone, γ-butyrolactone, δ-valerolactone, or ε-caprolactone. Representative diols of the formula HO—(CH₂)ₙ—NHCOO—(CH₂)ₘ—OH where n and m are each integers of 2 to 6 are made by the reaction of the same aminoalcohols just mentioned with cyclic carbonates of the formula

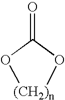

such as ethylene carbonate. Bis-amide diols of the formula HO—A—NHCO—B—CONH—A—OH are prepared by the reaction of a diacid, optionally in activated form, such as the diacyldihalide, with two equivalents of a hydroxy-amine. Other methods of preparation of the diols of the formula HO—R³—OH are known in the art.

Preparation of the Block Copolymers

The diblock copolymers of formula X are prepared in a two-step synthesis.

In the first step, a PEG lower alkyl ether of the formula R¹—[OCH₂CH₂]_{f}—OH, where R¹ is C₁₋₄ alkyl (an RPEG), is reacted with an excess of a diketene acetal of formula III or formula IV:

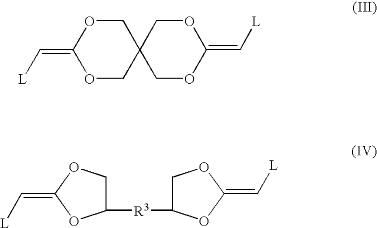

to form an intermediate of formula VII or formula VIII:

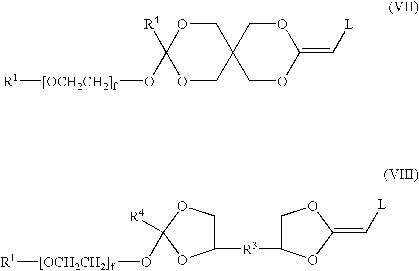

In the second step, a diol of the formula HO—R⁵—OH, HO—R⁶—OH, HO—R⁷—OH, or a mixture thereof, is reacted with the solution of the first step (containing the intermediate of formula VII or VIII and the excess diketene acetal) to extend the POE block, thereby forming the diblock copolymer of formula I.

Since the diketene acetal and the diol react in a 1:1 ratio to form the POE block of the diblock copolymer, the quantities of the RPEG, the diketene acetal, and the diol are chosen so that the molar amount of diketene acetal is equal to the sum of the molar amounts of the RPEG and the diol.

The value of f in the PEG block, i.e. the length of the PEG block, is determined by the RPEG chosen. The value of g in the POE block, i.e. the length of the POE block, is determined by the molar quantity of diol relative to the molar quantity of RPEG: the greater the molar quantity of diol (assuming that the diketene acetal is present in at least an equimolar quantity), the longer is the POE block.

The triblock copolymers of formula Y are also formed in a two-step synthesis.

In the first step, an excess of the diketene acetal of formula III or formula IV is reacted with a diol of the formula HO—R¹—OH, HO—R²—OH, or HO—R³—OH, or a mixture thereof, to form a POE block which is terminated at each end with a diketene acetal unit, giving an intermediate of formula IX or formula X:

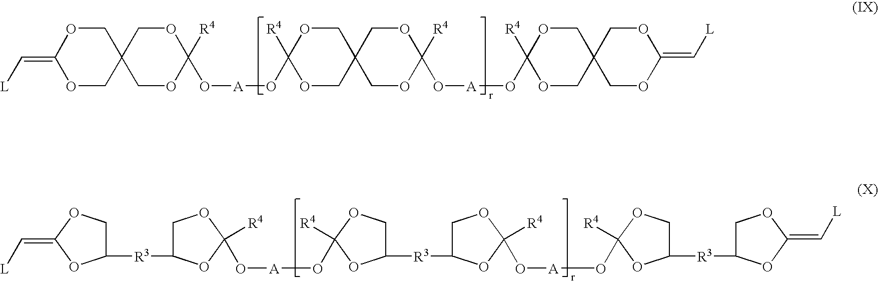

where r is g-2.

In the second step, the intermediate of formula IX or formula X is reacted with two equivalents of PEG or an RPEG to form the triblock copolymer of formula Y.

Since the diketene acetal and the diol react in essentially a 1:1 ratio to form the POE block of the triblock copolymer, but diketene acetal termination of the POE block is desired, the quantities of the diketene acetal and the diol are chosen so that the molar amount of diketene acetal is slightly greater than the molar amount of the diol. The molar ratio of PEG/RPEG to POE block should be approximately 2:1, but an excess of PEG/RPEG may be used, as it may be easily separated from the polymer after completion of the reaction.

The values of f and h for the PEG blocks are determined by the PEG/RPEG chosen. Typically f and h are the same, when a single PEG/RPEG is used; but if two or more PEGs/RPEGs of different lengths are used, then mixtures of copolymers containing varying PEG block lengths can be obtained, and these mixtures may be separated if desired, by such molecular weight fractionation techniques as gel permeation chromatography. The value of g for the POE block is determined primarily by the ratio of the diketene acetal to the diol used to form the POE.

The triblock copolymers of formula Z are also formed in a two-step synthesis.

In the first step, a PEG of the formula H—[OCH₂CH₂]ₕ—OH is reacted with an excess of a diketene acetal of formula III or formula IV to form an intermediate of formula XI or formula XII:

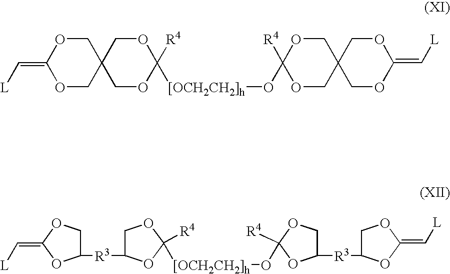

In the second step, a diol of the formula HO—R⁵—OH, HO—R⁶—OH, or HO—R⁷—OH, or a mixture thereof, is reacted with the solution of the first step (containing the intermediate of formula XI or formula XII and the excess diketene acetal) to extend the POE blocks, thereby forming the triblock copolymer of formula Z.

Since the diketene acetal and the diol react in a 1:1 ratio to form the POE blocks of the diblock copolymer, the quantities of the PEG, the diketene acetal, and the diol are chosen so that the molar amount of diketene acetal is equal to the sum of the molar amounts of the PEG and the diol.

The value of h for the PEG block is determined by the PEG chosen. The values of g and j for the POE blocks are determined by the molar quantity of diol relative to the molar quantity of PEG: the greater the molar quantity of diol (assuming that the diketene acetal is present in at least an equimolar quantity), the longer are the POE blocks. Typically the POE blocks will be of equal lengths, on average.

In an alternative synthesis of the triblock copolymer of formula Z, POE blocks terminated with diketene acetal units (intermediates of formula IX and formula X are prepared, and reacted with 0.5 molar equivalent of PEG to terminate each end of the PEG with the POE blocks.

In any of the syntheses in which the copolymers may have an unreacted diketene acetal terminal group, the copolymer may be reacted with a hydroxy-containing compound, such as a C₁-C₄ alcohol, to terminate the copolymer with alkoxy units; and such alkoxy-terminated copolymers are included within the scope of the invention. The hydroxy-containing compound, especially a C₁-C₄ alcohol, may be employed in excess and the unreacted excess easily separated during purification of the polymer.

Suitable reaction conditions for the formation of the copolymers are those conditions well known for the formation of poly(ortho esters), such as are described in U.S. Pat. No. 5,968,543 and the other documents cited in the BACKGROUND OF THE INVENTION. Typically, the reaction takes place in a polar aprotic solvent, such as those solvents mentioned previously for the preparation of the α-hydroxy acid containing diols, and ethers, especially tetrahydrofuran. A catalyst may be used if desired or necessary, and may be selected from those catalysts known to the art for the formation of orthoesters. Suitable such catalysts include iodine/pyridine, strong acids such as poluenesulfonic acid; Lewis acids, such as boron trichloride etherate, boron trifluoride etherate, tin oxychloride, phosphorus oxychloride, zinc chloride, phosphorus pentafluoride, antimony pentafluoride, stannic chloride, and the like; and Brønsted acids, such as polyphosphoric acid, polystyrenesulfonic acid, and the like. A particularly suitable catalyst is p-toluenesulfonic acid. A typical amount of catalyst used is about 0.2% by weight relative to the diketene acetal, though quantities between 0.005% and 2% may be used.

Suitable reaction temperatures are from room temperature to the boiling point of the solvent used, for example, between 20 C. and 70° C.; and suitable reaction times are between a few minutes and 48 hours, typically between 15 minutes and 24 hours.

Once the formation of the block copolymer is complete, the copolymer can be isolated by precipitation in a non-polar aprotic solvent such as hexane. Typically, the reaction mixture containing the copolymer (which may be cooled before the addition) is added slowly to about ten volumes of the rapidly stirred solvent at room temperature. The precipitated block copolymer may be collected by filtration, decantation, or other suitable method, washed to remove unreacted monomers or other contaminants, and dried, typically in a vacuum oven at a temperature below its melting point.

The bioerodibility of a block copolymer of this invention is determined by two factors: first, the extent to which the copolymer will dissolve/become suspended intact in an aqueous medium, the solubility of the copolymer; and second, the extent to which the copolymer, or, to be more precise, the POE block(s), will degrade in the environment to which it is exposed. The speed of degradation of the POE block(s) of the copolymer in an aqueous environment is determined by the hydrophilicity of the copolymer and by the proportion of α-hydroxy acid ester groups, if present, in the block(s), with greater bioerodibility being achieved by inclusion of a greater proportion of diols of the formula HO—R¹—OH in the diol mixture used to form the POE block(s).

Uses of the Block Copolymers of this Invention

While the block copolymers of this invention will find utility in any of the uses for which biodegradable polymers are useful, including such uses as vehicles for the sustained release of active agents, orthopedic implants, degradable sutures, and the like, they will also find particular utility in applications where their nature as block copolymers having both hydrophobic and hydrophilic blocks confers a special benefit, and these uses will be addressed in greater detail, since a person of ordinary skill in the art will be well acquainted with the uses of biodegradable polymers and will have no difficulty, having regard to the skill of the art and this disclosure, in adapting the block copolymers of this invention to such uses.

Micellar System for Targeting of Tissues with EPR (Tumors and Inflamed Tissues)

Polymers useful as micellar delivery systems can be prepared by forming diblock, AB, or triblock, ABA or BAB, copolymers comprising a hydrophilic poly(ethylene glycol) A block and a hydrophobic poly(ortho ester) B block.

When such block copolymers are placed in water, in which the poly(ethylene glycol) block is soluble and the poly(ortho ester) block is insoluble, the block copolymer chains will spontaneously self-aggregate to form micellar structures. The hydrodynamic diameter of such micelles, which may be determined by methods such as dynamic light scattering, will be in the order of 10-30 nm. As may be determined by methods such as static light scattering, such micelles will contain several hundred polymer chains. The micelles will undergo a secondary, reversible association, giving particles of an average diameter of about 100 nm. While such micelles are too large to be excreted by the kidneys, individual block copolymers are not. Further, since the poly(ortho ester) segments can be made to be biodegradable, facile renal excretion will take place.

The major utility of such micellar systems resides in their ability to entrap and solubilize hydrophobic drugs in the hydrophobic core. Such entrapment is easily carried out in a number of ways. Thus, the drug can be added to the aqueous solution containing micelles and incorporated by simple stirring, by heating to moderate temperatures, or by ultrasonication. The micelles are efficient carriers for a variety of hydrophobic or insoluble active agents, and are particularly suitable as carriers for anticancer agents, which will accumulate in the tumor by an endocytotic process.

Efficient entrapment of hydrophobic drugs requires a highly hydrophobic core. Using AB, ABA, or BAB block copolymers where the hydrophobic B block forms a biodegradable, highly hydrophobic poly(ortho ester) core will allow preparation of systems with significantly enhanced entrapment efficiency relative to other biodegradable segments such as poly(L-lactic-co-glycolic acid) copolymers.

While any of the anticancer agents that can form micellar complexes are suitable for this use, anticancer agents that are particularly suitable for micellar tumor targeting are those with low water solubility or high aromatic content, such as the anthracycline antibiotics (e.g. doxorubicin, daunorubicin, and epirubicin), mitomycin C, paclitaxel and its analogs (e.g. docetaxol), platinum analogs (e.g. cisplatin and carboplatin), and the like. Other agents may include anticancer proteins, such as neocarzinostatin, L-asparaginase, and the like, and photosensitizers used in photodynamic therapy. Similarly, while any of the anti-inflammatory agents that can form micellar complexes are suitable for this use, anti-inflammatory agents that are particularly suitable for micellar targeting are those with low water solubility or high aromatic content, such as the anti-inflammatory steroids (e.g., cortisone, hydrocortisone, dexamethasone, prednisone, prednisolone, beclomethasone, betamethasone, flunisolide, fluocinolone acetonide, fluocinonide, triamcinolone, and the like) and the non-ionized NSAIDs (e.g., naproxen, nabumetone, ketoprofen, mefenamic acid, fenbufen, piroxicam, meloxicam, celecoxib, rofecoxib, and the like).

Bioerodible Block Copolymer Matrix for Controlled Drug Delivery

In the block copolymers of this invention, phase separation will occur where domains of the B block form within the continuous A-phase or vice versa. Such phase-separated material will have unique and useful thermal properties. Specifically, unlike poly(ortho esters) containing short segments of PEG within the poly(ortho ester), which when heated will gradually soften, PEG/POE AB, ABA, or BAB block copolymers have relatively sharp melting points. Further, while poly(ortho esters) containing short segments of poly(ethylene glycol) that have low softening temperatures have very poor mechanical properties, the copolymers of this invention, even those having very low melting temperatures, will retain mechanical properties suitable for use as implants.

To use the copolymer as a sustained-release vehicle, the active agent must be incorporated into a matrix of the copolymer or encapsulated within a capsule (or a “microcapsule” or “nanocapsule”, as those terms are sometimes used) of the copolymer. Methods for the preparation of sustained-release dosage forms using biodegradable polymers are well known in the art, as discussed in the references cited in the BACKGROUND OF THE INVENTION section of this application, and in other references familiar to those of ordinary skill in the art; so that a person of ordinary skill in the art would have no difficulty, having regard to that skill and this disclosure, in preparing sustained-release formulations using the copolymer of this invention. Suitable active agents include therapeutic agents such as pharmaceutical or pharmacological active agents, e.g. drugs and medicaments, as well as prophylactic agents, diagnostic agents, and other chemicals or materials useful in preventing or treating disease. The compositions of this invention are particularly useful for the therapeutic treatment of humans and other mammals, but may also be used for other animals. In addition, the sustained-release compositions of this invention may also be used for the release of cosmetic and agricultural agents, or for the release of biocides, such as fungicides or other pesticides, into an environment where prolonged release of the active agent is desired.

In the case of matrix formulations, the copolymer is first mixed with the active agent. High homogeneity may be achieved by mixing the polymer in its heat softened state with the active agent, followed by lowering the temperature to harden the composition. Alternatively, the copolymer can be dissolved in an appropriate casting solvent, such as tetrahydrofuran, methylene chloride, chloroform, or ethyl acetate, and the active agent can then be dispersed or dissolved in the copolymer solution, followed by evaporating the solvent to achieve the finished composition. Another method is grinding a solid copolymer material into powder which is then mixed with a powdered active agent. The active agent may also be incorporated into the mixture of monomers before polymerization provided that it is stable under the polymerization conditions and does not interfere with the polymerization reaction.

If the active agent is one that is unstable at elevated temperatures (e.g. above 40° C.), or in the presence of organic solvents or organic solvent/water mixtures, such as a protein, then special preparation techniques may be required to minimize the exposure of the active agent to damaging conditions. Such techniques are disclosed in, for example, U.S. Pat. Nos. 5,620,697, which discloses ultrasonic melting to form matrix-type pharmaceutical compositions, and 5,518,730, which discloses melt-spinning, both of which techniques are designed to minimize the exposure of the polymer and active to elevated temperatures. Other methods are disclosed in the documents cited elsewhere in this application.

An alternate method for the incorporation and release of sensitive therapeutic agents is to use bioerodible copolymers that have physical properties tailored for this incorporation. For example, the copolymer may be chosen so that it is semi-solid and has an ointment-like consistency, rather than being fully solid. Thus, a copolymer may be chosen that has a very high viscosity at normal body temperature of 37° C. so that little if any deformation takes place at that temperature. However, the viscosity of the copolymer may decrease substantially at temperatures no higher than 45° C., or preferably by 40° C., so that injection of the material may be possible at a temperature at which the active agent retains its activity.

The composition obtained from any of the above methods can be readily processed into a variety of shapes and forms for implantation, insertion or placement on the body or into body cavities or passageways. For example, the copolymer composition may be injection molded, extruded or compressed into a thin film or made into devices of various geometric shapes or forms such as flat, square, round, cylindrical, tubular, disc, ring and the like. Rod- or pellet-shaped devices may be implanted through a trocar, such as is known for Norplant® implants, and these or other shapes may be implanted by minor surgical procedures. Alternatively, a device may be implanted following a major surgical procedure such as tumor removal in the surgical treatment of cancer. The implantation of polymer wafers containing anticancer agents is described, for example, in U.S. Pat. Nos. 5,626,862 and 5,651,986, and references cited therein; and the copolymers of this invention will find utility in such applications.

The polymer composition may also be injected by syringe subcutaneously or intramuscularly as particles of 0.1 μm to 1000 μm, preferably 0.5 μm to 200 m, and more preferably 1 μm to 150 μm suspended in a pharmaceutically acceptable injection base. Liquid vehicles useful for suspending the drug-copolymer composition for injection include isotonic saline solution or oils (such as corn oil, cottonseed oil, peanut oil and sesame oil) which, if desired, may contain other adjuvants.

Another injectable dosage form may be prepared from an active agent mixed in with a copolymer of the present invention which has an ointment-like consistency. Such a dosage form may be administered by injection with or without a solvent.

The copolymer composition administered by either injection or implantation undergoes bioerosion in the body into non-toxic and non-reactive materials. By controlling the number of hydrolysable bonds in the polymer, the active agent may be released at a desired rate. Implants prepared from the present copolymers in which the copolymer constitutes the matrix containing an active agent also have the advantage that they do not require removal because of the bioerodibllity of the copolymer.

In some cases, particles with cores of the pure active agent coated with various thicknesses of the present copolymer may be preferred for sustained delivery of the active agent. Coating or encapsulation of discrete particles of the active agent may be accomplished by conventional methods which are all well-known to the person skilled in the art. For example, finely divided drug particles may be suspended in a solvent system (in which the drug is not soluble) containing the dissolved copolymer and other excipients, followed by spray drying. Alternatively, the drug particles may be placed in a rotating pan or a fluid-bed dryer and the copolymer dissolved in a carrier solvent is sprayed onto the drug particles until a suitable coating quantity is deposited on the particles to give a desired thickness. The coating may also be achieved by suspending the drug particles in a solvent system containing the dissolved copolymer followed by adding to the suspension a non-solvent causing the copolymer to precipitate and form a coating over the drug particles.

For the sustained release compositions, because the active agent will be released over a controlled period of time, the agent usually is present in an amount which is greater than the conventional single dose. The relative proportions of the active agent and the copolymer can vary over a wide range (e.g., 0.1 to 50 weight percent) depending on the therapeutic agent and the desired effect.

Sustained compositions of cosmetic and agricultural agents may also be prepared by any one of the methods as described above, using the copolymers of the present invention.

The solid copolymers are also useful for a variety of orthopedic applications. For example, they can be used as fracture fixation devices for repair of osteochondral defects, ligament and tendon reconstructions and bone substitutes. In addition, the fact that the present copolymers permit simultaneous selection of both a desired level of their mechanophysical state and a desired rate of bioerodibility, also renders them attractive as grafts or scaffolds on which cells can be cultured in vitro prior to implantation to regenerate tissues. Tissues which can be regenerated using this approach include but are not limited to bone, tendon, cartilage, ligaments, liver, intestine, ureter and skin tissues. For example, the copolymers may be used to regenerate skin for patients with burns or skin ulcers. Cartilages may be repaired by first isolating chondrocytes from a patient (or a donor), allowing them to proliferate on the scaffolds prepared from the present copolymer and re-implanting the cells in the patient.

The copolymer scaffolds or implants may further contain other biologically active substances or synthetic inorganic materials such as reinforcing filler material for enhancing the mechanical properties of the scaffolds or implants (e.g. calcium sodium metaphosphate fibers), antibiotics, or bone growth factors to induce and/or promote orthopedic restoration and tissue regeneration.

The compositions are also stable. The release rates of the active agent are not affected by irradiation for sterilization.

Particular Compositions and Their Uses

Exemplary compositions of this invention, and their uses, include:

(1) compositions containing local anesthetics, optionally in combination with glucocorticosteroids such as dexamethasone, cortisone, hydrocortisone, prednisone, prednisolone, beclomethasone, betamethasone, flunisolide, fluocinolone acetonide, fluocinonide, triamcinolone, and the like, for the prolonged relief of local pain or a prolonged nerve blockade. This use is discussed further below;

(2) compositions containing cancer chemotherapeutic agents, such as those listed above under “Active Agents”, for deposition by syringe or by injection into tumors or operative sites from which a tumor has been ablated, for tumor control or treatment and/or the suppression of regrowth of the tumor from residual tumor cells after ablation of the tumor;

(3) compositions containing progestogens, such as flurogestone, medroxyprogesterone, norgestrel, norgestimate, norethindrone, and the like, for estrus synchronization or contraception;

(4) compositions containing antimetabolites such as fluorouracil and the like, as an adjunct to glaucoma filtering surgery; compositions containing antiangiogenic agents such as combrestatin, contortrostatin, and anti-VEGF agents, for the treatment of macular degeneration and retinal angiogenesis; and other compositions for the controlled release of ophthalmnic drugs to the eye;

(5) compositions containing therapeutic polypeptides (proteins), such as insulin, luteinizing hormone releasing factor antagonists, and the like, for the controlled delivery of these polypeptides, avoiding the need for daily or other frequent injection;

(6) compositions containing anti-inflammatatory agents such as the NSAIDs, e.g., ibuprofen, naproxen, COX-1 or COX-2 inhibitors, and the like, or anti-inflammatory steroids, for deposition by injection into inflamed tissue or intra-articular injection;

(7) compositions containing antibiotics, for the prevention or treatment of infection, especially for deposition into surgical sites to suppress post-operative infection, or into or on wounds, for the suppression of infection (e.g. from foreign bodies in the wound);

(8) compositions containing morphogenic proteins such as bone morphogenic protein; and

(9) compositions containing DNA or other polynucleotides, such as antisense oligonucleotides.

Delivery of Controlled-Release Local Anesthetics by Injection

Local anesthetics induce a temporary nerve conduction block and provide pain relief which lasts from a few minutes to a few hours. They are frequently used to prevent pain in surgical procedures, dental manipulations or injuries.

The synthetic local anesthetics may be divided into two groups: the slightly soluble compounds and the soluble compounds. Conventionally, the soluble local anesthetics can be applied topically and by injection, and the slightly soluble local anesthetics are used only for surface application. The local anesthetics conventionally administered by injection can also be divided into two groups, esters and non-esters. The esters include (1) benzoic acid esters (piperocaine, meprylcaine and isobucaine); (2) p-aminobenzoic acid esters (procaine, tetracaine, butethamine, propoxycaine, chloroprocaine); (3) m-aminobenzoic acid esters (metabutethamine, primacaine); and (4) p-ethoxybenzoic acid esters (parethoxycaine). The non-esters are largely anilides (amides), and include bupivacaine, lidocaine, mepivacaine, pyrrocaine and prilocaime.

Many of the local anesthetics are conventionally used in the form of their acid addition salts, as this provides solubility in aqueous injection media. However, because the presence of the large amount of acid within such a local anesthetic acid addition salt will result in more rapid degradation of the poly(ortho esters) and release of the local anesthetic, it is generally desirable to use the local anesthetics in free base form, or with only a small proportion of the acid addition salt present (addition of small quantities of the acid addition salt may provide enhanced release if desired).

The semi-solid injectable form of a local anesthetic of the present invention is prepared by incorporating the local anesthetic into the delivery vehicle in a manner as described above. The concentration of the local anesthetic may vary from 1-60 wt. %, preferably 5-30 wt. %, e.g., about 10 wt. %. The semi-solid composition is then filled into a syringe with a 18-25 gauge needle, and injected into sites that are painful or to be subjected to surgical procedures. The semi-solid injectable composition of the present invention can be used for controlled delivery of both slightly soluble and soluble local anesthetics.

Because the duration of action of a local anesthetic is proportional to the time during which it is in actual contact with nervous tissues, the present injectable delivery system can maintain localization of the anesthetic at the nerve for an extended period of time which will greatly prolong the effect of the anesthetic.

A number of authors, including in U.S. Pat. No. 6,046,187 and related patents, have suggested that the co-administration of a glucocorticosteroid may prolong or otherwise enhance the effect of local anesthetics, especially controlled-release local anesthetics; and formulations containing a local anesthetic and a glucocorticosteroid, and their uses for controlled release local anesthesia, are within the scope of this invention.

### EXAMPLES

Preparation 1: Preparation of a diketene acetal of formula IV, 3,9-di(ethylidene)-2,4,8,10-tetraoxaspiro[5.5]undecane, DETOSU

A 3-liter, 3-necked flask fitted with a mechanical stirrer, argon inlet tube, thermometer and rubber septum was charged with 1.2 L ethylenediamine. The flask was cooled with ice water and the contents kept at about 8° C. under an argon atmosphere. A hexane solution of n-butyllithium, 130 g (2 mol n-BuLi), was added over one hour through a stainless steel U-tube pushed through the rubber septum, using carefully controlled argon pressure. Next, a mixture of 3,9-divinyl-2,4,8,10-tetraoxaspiro[5.5]undecane, 530 g (2.5 mol), (available from Aldrich Chemical Company, Inc., Milwaukee, Wis. USA) and 0.5 L ethylenediamine was cooled to 8° C. and added to the flask. After stirring at 8° C. for 3 hours, the reaction mixture was poured into 3 L of ice water with vigorous stirring. The aqueous mixture was extracted twice with 1 L portions of hexane. The combined hexane extracts were washed three times with 1 L portions of water, dried over anhydrous magnesium sulfate and filtered under suction. The filtrate was evaporated to dryness on a rotary evaporator to give crude material (413 g, 78%) containing 90% 3,9-di(ethylidene)-2,4,8,10-tetraoxaspiro[5.5]undecane.

The crude 3,9-di(ethylidene)-2,4,8,10-tetraoxaspiro[5.5]undecane product was dissolved in 2 L hexane containing 10 mL triethylamine and the solution was placed in a 4 L filter flask, sealed, and stored in a freezer at −20° C. for two days. The crystals thus formed were collected by basket centrifugation at −5° C. under an argon atmosphere. Distillation of the brownish product through a 12-inch Vigreaux column at reduced pressure gave 313 g (61% yield) 3,9-di(ethylidene)-2,4,8,10-tetraoxaspiro[5.5]undecane as a colorless liquid, boiling point 82° C. (0.1 Torr), which crystallized at room temperature, with a melting point of 30° C. and a characteristic infrared absorption band at 1700 cm⁻¹.

Preparation 2: Preparation of a diol where A is R⁵

Under anhydrous conditions, 14.42 g (100 mmol) 1,4-cyclohexanedimethanol and 11.6 g (100 mmol) glycolide were weighed into a 100 mL round bottom flask. The flask was stoppered with a rubber septum, then heated in an oil bath at 180° C. for 24 hours. The product, 4-(hydroxymethyl)-cyclohexylmethoxycarbonylmethyl hydroxyacetate, was obtained as a viscous oil.

Using an analogous procedure with a 2:1 ratio of tetraethylene glycol, H—(OCH₂CH₂)₄—OH, to glycolide there was obtained 2-{2-[2-(2-hydroxyethoxy)ethoxy]ethoxy}ethyl hydroxyacetate.

### Example 1

Preparation of Diblock Copolymers of Formula X

Under anhydrous conditions, 20 g (10 mmol) PEG 2000 mono-methyl ether (MPEG 2000) and 21.23 g (100 mmol) 3,9-di(ethylidene)-2,4,8,10-tetraoxaspiro[5.5]undecane (DETOSU) were weighed into a 250 mL flask and dissolved in 40 mL tetrahydrofuran. A solution of p-toluenesulfonic acid in tetrahydrofuran (0.05 mL, 20 mg/mL) was added to the MPEG 2000/DETOSU solution to initiate the reaction between the MPEG 2000 and the DETOSU, and the reaction mixture was stirred for about 20 min. 1,4-Cyclohexanedimethanol (13.20 g, 91.5 mmol) and 0.266 g (1 mmol) 2-{2-[2-(2-hydroxyethoxy)ethoxy]ethoxy} ethyl hydroxyacetate in 40 mL tetrahydrofuran were added to the flask, followed by another 0.05 mL of the p-toluenesulfonic acid solution. The reaction mixture was stirred for about 30 min, and then added dropwise to about 1 L of hexane with vigorous stirring, precipitating the diblock copolymer product, which was separated by filtration and dried in a vacuum oven.

Using similar procedures, diblock copolymers were prepared from the starting materials given in the table below:

| DETOSU (mmol) | M-PEG (Mw) (mmol) | CDM (mmol) | TEG (mmol) | TEG-G1 (mmol) |
|---|---|---|---|---|
| 21.23 g (100) | 10 g (2000) (5) | 12.79 g (90) | 0.976 g (6.5) | 0.266 g (1) |
| 21.23 g (100) | 25 g (5000) (5) | 12.79 g (90) | 0.976 g (6.5) | 0.266 g (1) |
| 21.23 g (100) | 35 g (5000) (7) | 12.79 g (90) | 0.676 g (4.5) | 0.532 g (2) |

DETOSU = 3,9-di(ethylidene)-2,4,8,10-tetraoxaspiro[5.5]undecane
M-PEG = poly(ethylene glycol) monomethyl ether, with weight average molecular weight in parentheses
CDM = 1,4-cyclohexanedimethanol
TEG = 2-{2-[2-(2-hydroxyethoxy)ethoxy]ethoxy}ethanol
TEG-G1 = 2-{2-[2-(2-hydroxyethoxy)ethoxy]ethoxy}ethyl hydroxyacetate

### Example 2

Preparation of Triblock Copolymers of Formula Z

Under anhydrous conditions, 15 g (15 mmol) PEG 1000 and 21.23 g (100 mmol) 3,9-di(ethylidene)-2,4,8,10-tetraoxaspiro[5.5]undecane (DETOSU) were weighed into a 250 mL flask and dissolved in 40 mL tetrahydrofuran. A solution of p-toluenesulfonic acid in tetrahydrofuran (0.05 mL, 20 mg/mL) was added to the PEG 1000/DETOSU solution to initiate the reaction between the PEG 1000 and the DETOSU, and the reaction mixture was stirred for about 20 min. 1,4-Cyclohexanedimethanol (11.52 g, 79.9 mmol), 0.638 g (4.25 mmol) 2-{2-[2-(2-hydroxyethoxy)-ethoxy]ethoxy}ethanol and 0.226 g (0.85 mmol) 2-{2-[2-(2-hydroxyethoxy)ethoxy]ethoxy}ethyl hydroxyacetate in 40 mL tetrahydrofuran were added to the flask, followed by another 0.05 mL of the p-toluenesulfonic acid solution. The reaction mixture was stirred for about 30 min, and then added dropwise to about 1 L of hexane with vigorous stirring, precipitating the diblock copolymer product, which was separated by filtration and dried in a vacuum oven, giving a triblock POE-PEG-POE copolymer of 33,370 weight average molecular weight.

Using similar procedures, triblock copolymers of formula Z were prepared from the starting materials given in the table below:

| DETOSU (mmol) | PEG (Mw) (mmol) | CDM (mmol) | TEG (mmol) | TEG-Gl (mmol) |
|---|---|---|---|---|
| 21.23 g (100) | 30 g (1000) (30) | 9.95 g (69) | 0 | 0.266 g (1) |
| 21.23 g (100) | 15 g (4600) (3.26) | 13.115 g (90.94) | 0.726 g (4.837) | 0.257 g (0.967) |
| 21.23 g (100) | 23 g (4600) (5) | 13.56 g (94) | 0 | 0.266 g (1) |
| 21.23 g (100) | 46 g (4600) (10) | 12.84 g (89) | 0 | 0.266 g (1) |

DETOSU = 3,9-di(ethylidene)-2,4,8,10-tetraoxaspiro [5.5]undecane
PEG = poly(ethylene glycol), with weight average molecular weight in parentheses
CDM = 1,4-cyclohexanedimethanol
TEG = 2-{2-[2-(2-hydroxyethoxy)ethoxy]ethoxy}ethanol
TEG-Gl = 2-{2-[2-(2-hydroxyethoxy)ethoxy]ethoxy}ethyl hydroxyacetate

Other copolymers of formula X, Y, and Z are similarly prepared.

### Example 3

Solubility of the Copolymers

The copolymer of Example 1, 100 mg, is dissolved in 2 mL acetone, and 20 mL phosphate-buffered saline, pH 7.4, is added. No precipitation of the copolymer is observed. The solution is placed under aspirator vacuum at room temperature to remove the acetone, and the copolymer remains in solution.

Other copolymers of formulae X, Y, and Z show similar solubility.

### Example 4

Solubilization of Hydrophobic/Water-Insoluble Active Agents

The copolymer of Example 1, 100 mg, is dissolved in 2 mL acetone, and the solution added to a solution of 7.7 mg hydrocortisone in 2 mL acetone. The combined acetone solutions are added to 5 mL phosphate-buffered saline, pH 7.4, the acetone removed under vacuum, and the aqueous solution filtered through a 0.45 μm filter. The aqueous solution is found to have a hydrocortisone concentration greater than the water solubility of hydrocortisone of 0.28 mg/mL, indicating tricellar encapsulation and solubilization of the hydrocortisone by the copolymer.

Other copolymers of formula X, Y, and Z show similar solubilization of hydrophobic/water-insoluble active agents.

### Example 5

Bioerodibility of the Copolymers

The copolymer of Example 1 is pressed at 48° C. and 1000 mPa into a slab 0.6 mm thick, and the slab then cut into wafers approximately 6 mm×6 mm. The wafers are weighed, and then placed into phosphate-buffered saline, pH 7.4, at 37° C.; and the weight loss of the wafers measured as a function of time. The copolymer is bioerodible as shown by loss in weight over time.

Other copolymers of formulae X, Y, and Z show similar bioerodibility.

While this invention has been described in conjunction with specific embodiments and examples, it will be evident to one of ordinary skill in the art, having regard to this disclosure, that equivalents of the specifically disclosed materials and techniques will also be applicable to this invention; and such equivalents are intended to be included within the following claims.

What is claimed is:

1. A block copolymer of formula X, formula Y, or formula Z:
R¹—[OCH₂CH₂]_{f}—[POE]_{g}—H (X),
R¹—[OCH₂CH₂]_{f}—[POE]_{g}—[OCH₂CH₂]ₕ—OR² (Y),
H—A—[POE]_{g}—[OCH₂CH₂]ₕ—[POE]ⱼ—H (Z),
where:
R¹ is C₁₋₄ alkyl;
R² is C₁₋₄ alkyl;
f and h are independently an integer from 2 to 1000;
g and j are independently an integer from 2 to 200;
POE is a poly(ortho ester) of formula I or formula II: 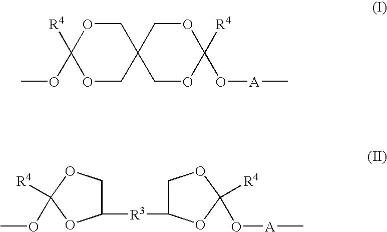
where:
R³ is a bond, —(CH₂)ₐ—, or —(CH₂)_{b}—O—(CH₂)_{c}—; where a is an integer of 1 to 10, and b and c are independently integers of 1 to 5;
R⁴ is a C₁₋₄ alkyl; and
each A is R⁵, R⁶, or R⁷, where
R⁵ is: 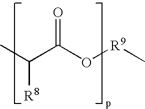
where:
p is an integer of 1 to 20;
R⁸ is hydrogen or C₁₋₄ alkyl; and
R⁹ is: 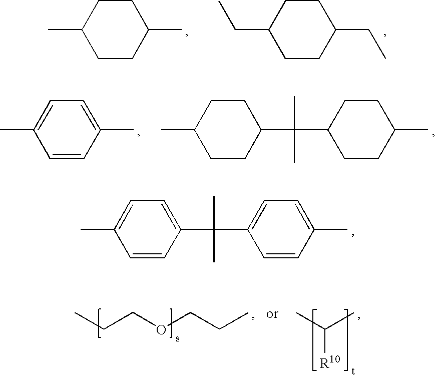
where:
s is an integer of 0 to 30;
t is an integer of 2 to 20; and
R¹⁰ is hydrogen or C₁₋₄ alkyl;
R⁶ is: 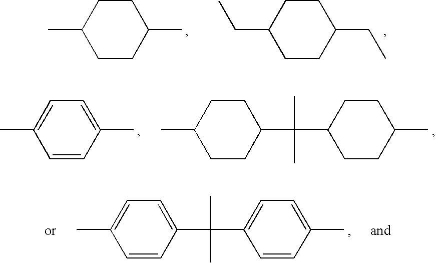
R⁷ is a diol containing at least one functional group independently selected from amide, imide, urea, and urethane groups;
where A is R⁷ in at least 0.1 mol % of the POE units when the poly(ortho ester) is of formula I.

2. The copolymer of claim 1 where at least 0.1% of the POE units are of formula II.

3. The copolymer of claim 2 where R¹ and R² are both methyl.

4. The copolymer of claim 2 where R⁴ is ethyl.

5. The copolymer of claim 2 where p is 1 or 2, and R⁸ is methyl.

6. The copolymer of claim 2 where HO—R⁶—OH is 1,4-cyclohexanedimethanol.

7. The copolymer of claim 2 which is a compound of formula Z.

8. The copolymer of claim 7 where h is an integer from 10 to 500, and g and j are independently an integer from 5 to 100.

9. The copolymer of claim 1 which is a compound of formula X.

10. The copolymer of claim 9 where f is an integer from 10 to 500 and g is an integer from 5 to 100.

11. The copolymer of claim 2 where at least 50% of the POE units are of formula II.

12. The copolymer of claim 11 where 100% of the POE units are of formula II.

13. A micellar pharmaceutical composition for the delivery of a hydrophobic or water-insoluble active agent, comprising the active agent physically entrapped within but not covalently bonded to a drug carrier comprising a block copolymer of claim 1.

14. A micellar pharmaceutical composition for the delivery of a hydrophobic or water-insoluble active agent, comprising the active agent physically entrapped within but not covalently bonded to a drug carrier comprising a block copolymer of claim 2.

15. The composition of claim 14 where the active agent is an anticancer agent.

16. The composition of claim 14 where the active agent is an anti-inflammatory agent.

17. A composition for the sustained release of an active agent, comprising the active agent dispersed in a matrix comprising the block copolymer of claim 1.

18. A composition for the sustained release of an active agent, comprising the active agent dispersed in a matrix comprising the block copolymer of claim 2.

19. The composition of claim 18 where the active agent is an anticancer agent.

20. The composition of claim 18 where the active agent is an anti-inflammatory agent.
